(19) United States
(12) Patent Application Publication
(10) Pub. No.: US 2021/0087203 A1
(43) Pub. Date: Mar. 25, 2021

(54) PROCESS FOR PREPARING SPIROCYCLIC COMPOUNDS

(71) Applicant: University of Huddersfield, Huddersfield, Yorkshire (GB)

(72) Inventor: Joseph Sweeney, Huddersfield, Yorkshire (GB)

(21) Appl. No.: 16/480,537

(22) PCT Filed: Apr. 10, 2017
(86) PCT No.: PCT/GB2017/051001

§ 371 Date: Jul. 24, 2019

(30) Foreign Application Priority Data
Apr. 8, 2016 (GB) 1606003.0

(51) Int. Cl. C07D 493/10

(52) U.S. Cl. CPC C07D 493/10; C07B 53/00

(57) ABSTRACT

New spirocyclic ligands for use in metal catalysed asymmetric hydrogenation, hydroformylation, allylic substitution and a process for the production of the same from plant feedstocks.

The present invention relates to the catalytic production of spirocyclic compounds and processes including said catalysts.

Although the following description refers exclusively to the production of spirocyclic compounds the skilled person will appreciate that the present method is not limited to the production of such compounds and other heterocyclic compounds can be produced. In addition, plant derived precursors are utilised, however the person skilled in the art will appreciate that other conventional precursors could be used and the invention is not limited to as such.

Methods to access new heterocyclic fragments offer powerful creative possibilities to a range of chemistry end-users, including polymer, bulk chemical, biomedical and pharmaceutical industries. This is particularly true of heterocycles containing a high density of sp³-carbon centres, which are information-rich and offer precise spatial definition for chemical probes, chiral monomers and the like. Nonetheless, simple, catalytic routes to new heterocyclic cores are infrequently reported, and cores based on biomass-accessible subunits are also rare.

It is no exaggeration to state that metal-catalyzed carbon-carbon bond-forming reactions have revolutionized contemporary academic and industrial chemistry, and commercial products (including polymers, diagnostic materials, fine chemicals and active drug substances) are regularly prepared in bulk using the methods. Notwithstanding this, the financial, regulatory and environmental demands placed upon chemistry end-users have challenged contemporary catalysis researchers to deliver cleaner, more efficient and cost-effective methods for C—C bond formation; this has stimulated many research programmes focused on the use of less resource-intensive metals as mediators of C—C bond-forming processes, and iron catalysts have received particular attention. At the same time, the use of biomass-derived chemical feedstocks has been of increasing interest to the global chemical research community and this area will become increasingly significant for societal impact as fossil fuel stocks dwindle.

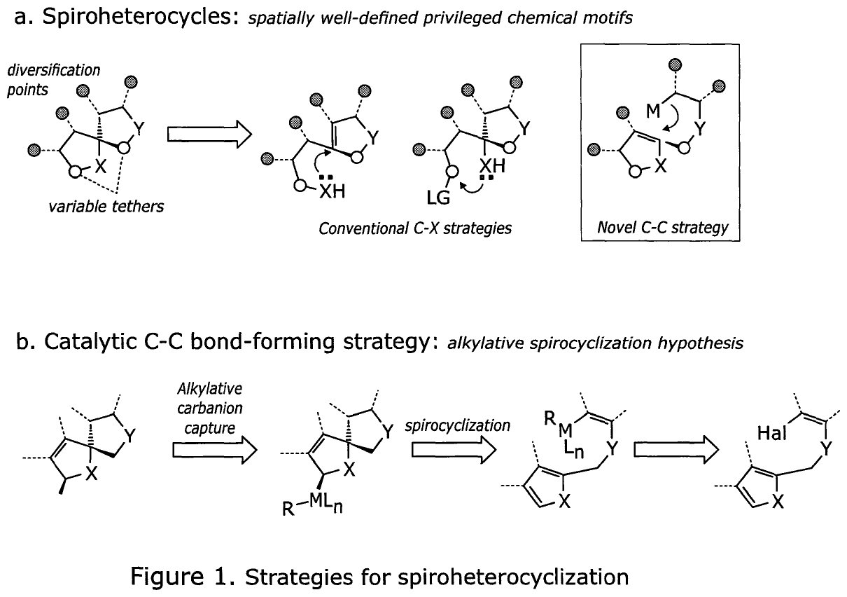

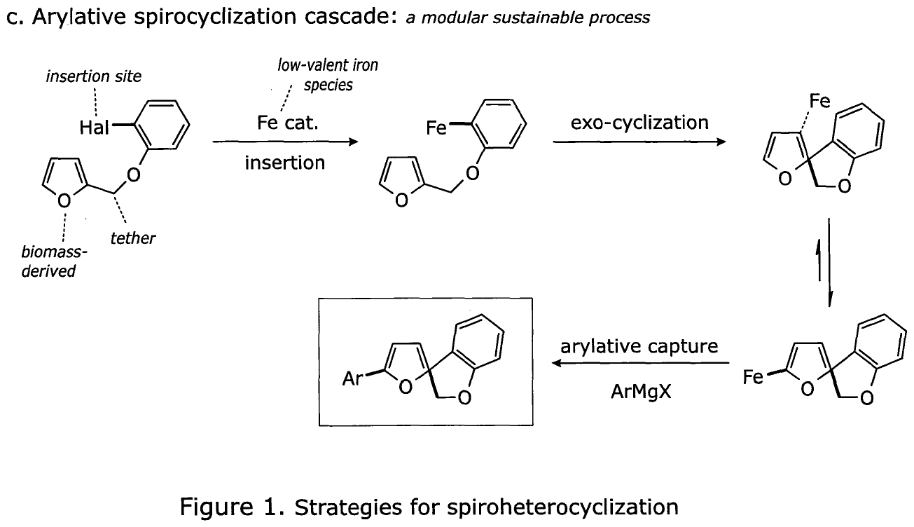

Alongside the increasing momentum to deliver sustainable processes, scientists are continually challenged by end-users to provide access to novel chemical architectures, such as novel heterocycles containing higher proportions of 3-D submotifs, and the development of methods to access chemical starting points containing increased levels of sp³ atoms has therefore been of great recent interest to researchers. Within this molecular class, small molecules bearing quaternary centres (in which there is maximum carbon diversity) are desirable but challenging targets and heterocycles which contain such motifs are currently accessed with difficulty. Spiroheterocycles, combining many of the chemical features desired by modern end-users, are known to be privileged substructures, but there are few general methods available to deliver the compounds. This is especially true for spiroethers and spiroamines, which are most often prepared by C—X bond-formation (FIG. 1, a)^{1,2}; we hypothesized that members of this 3-D heterocycle class could be accessed via a catalytic C—C bond-forming cascade (FIG. 1, b).

It is therefore an aim of the present invention to provide a new method or process by which to synthesis heterocycles.

It is a further aim of the present invention to provide a new route to spirocyclic compounds.

It is a yet further aim of the present invention to address the abovementioned problems.

In a first aspect of the invention there is provided a process suitable for the production of spirocyclic compounds, said process including the steps of;
introducing or mixing at least one halogenated heterocyclic compound with iron or an iron containing complex; and
introducing at least one organometallic compound.

Preferably the iron containing complex includes iron (III), iron in the 3+ oxidation state. Typically the iron or iron containing complex is in a catalytic amount. Further typically the process includes substantially 5 mol. % of iron (III) catalyst.

We report here a new entry to spirocyclic heterocycles, in which inexpensive iron(III)-catalysts mediate an unprecedented and highly stereoselective arylative cyclization cascade reaction of modular, plant-derived precursors.

In one embodiment the process uses any one or any combination of Fe(acac)₃, FeCl₃, Fe(dbm)₃ and/or the like.

Typically the organometallic compound is an aryl and/or alkyl magnesium halide (Grignard reagent).

Further typically the organometallic compound is an organozinc reagent.

In one embodiment the halogenated heterocycle is derived from plants or plant related compounds.

In a second aspect of the invention there is provided an iron(III)-catalyzed arylative spirocyclization.

Typically the spirocyclization is of at least one halogenated heterocyclic compound.

In a further aspect of the invention there is provided a spirocyclic ligand suitable for use in metal catalysed asymmetric transformations wherein said ligand has the general formula

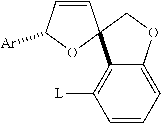

Typically Ar is an aromatic group. Further typically L is a Lewis base ligating group such as NR₂, PR₃ and/or the like.

Typically the ligands are for use in any one or any combination of metal catalysed asymmetric hydrogenation, hydroformylation, allylic substitution and/or the like.

We here report that the development of a 3-carbon novel and sustainable iron(III)-catalyzed arylative spirocyclization reaction (FIG. 1, c) which delivers these spiroheterocycles efficiently and stereoselectively.

### Results and Discussion

Our strategy as outlined in FIG. 1c sought to deliver a catalytic process in which a biomass-derived substrate would react under the influence of a low-valent iron species via a tandem spirocyclization/C—C bond-forming process. This would offer a sustainable and novel method for accessing novel heterocycles which would be information rich, due to the juxtaposition of planar motifs, heteroatoms and asymmetric sp³-carbon atoms.

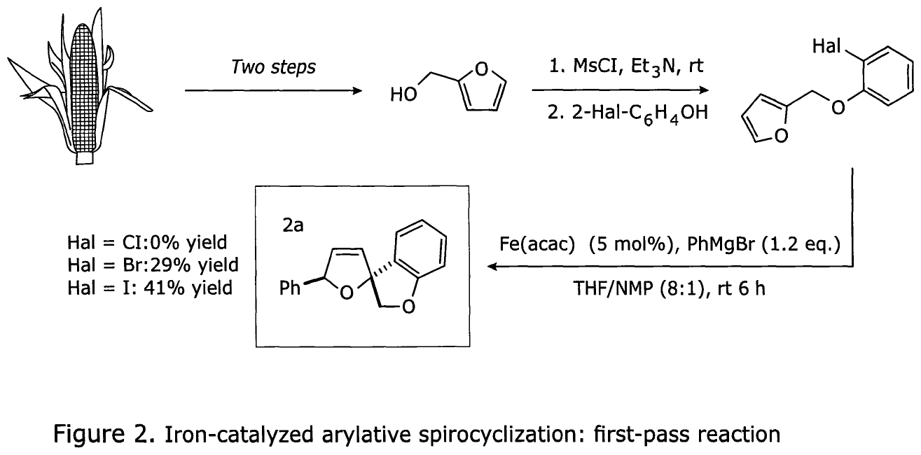

Landmark publications have identified key features of iron-mediated C—C bond forming reactions; our strategic premise was that in the presence of suitable additives a low-valent iron species (formed in situ by reaction of readily available Fe(III) salts with Grignard reagents) would mediate the spirocyclization cascade process shown in FIG. 1c. For the substrate, we targeted the use of furfuryl alcohol as a biomass-derived component, since it is available directly from plant sources (corn cobs) in a two-stage, highly scalable benchtop chemical process (FIG. 2). Activation followed by reaction with 2-halophenols gave the key lynchpin cyclization templates 1, which were reacted with phenyl magnesium bromide at room temperature in the presence of Fe(acac)₃ (5 mol %) in THF/NMP (8:1 ratio). Though reaction of the chloride was not productive, the corresponding bromide and iodide delivered phenylated spirocycle 2a (FIG. 2).

The tricyclic product 2a was delivered as a 6:1 ratio of diastereoisomers, in favour of the isomer in which the aryl rings are present in the trans-arrangement shown above (structural assignment based upon nOe experiments, Supplementary SX).

We built upon the encouraging initial results by next examining variation of solvent and the relative NMP content (Table 1); this screening process indicated that the optimum reaction medium was Et₂O/NMP (1:1, Table 1 entry 10).

TABLE 1
Influence of solvent on iron-catalyzed cascade

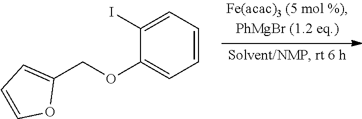 [SMILES: Ic1ccccc1OCc1ccco1]

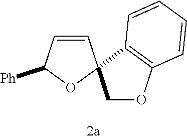

| | | Solvent: | Yield |
|---|---|---|---|
| Entry | Solvent | NMP | 2a (%) |
| 1 | THF | 8:1 | 41 |
| 2 | THF | 0:1 | 9 |
| 3 | THF | 1:3 | 36 |
| 4 | THF | 1:1 | 44 |
| 5 | THF | 3:1 | 27 |
| 6 | THF | 1:0 | 30 |
| 7 | Et₂O | 8:1 | 53 |
| 8 | Et₂O | 0:1 | 27 |
| 9 | Et₂O | 1:3 | 37 |
| 10 | Et₂O | 1:1 | 55 |
| 11 | Et₂O | 3:1 | 27 |
| 12 | Et₂O | 1:0 | 19 |
| 13 | DMF | 8:1 | 0 |
| 14 | DMPU | 8:1 | 0 |
| 15 | DMA | 8:1 | 12 |
| 16 | Dioxane | 8:1 | 0 |

The next phase of optimization examined a range of stable iron(III) catalysts in the arylative spirocyclization reaction (Table 2). It quickly transpired that the catalyst was essential for the reaction to take place (entry 1) and that iron(III) chloride was as effective a catalyst as Fe(acac)₃. When Grignard reagent was omitted, starting material was returned quantitatively.

At this stage of our studies we also noted that conversion and yields were consistently improved when an excess of Grignard reagents was employed, with 2.4 equivalents proving optimal. Iron complexes bearing bulkier ligands were less productive in the reaction (entries 7 and 8) but only Fe₂(SO₄)_{3 (entry} 9) proved ineffective, returning the starting material quantitatively.

TABLE 2
Influence of catalyst and Grignard stoichiometry

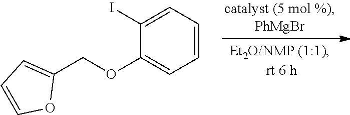 [SMILES: Ic1ccccc1OCc1ccco1]

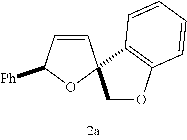

| | | PhMgBr | Yield 2a | Conversion |
|---|---|---|---|---|
| Entry | Catalyst | (eq.) | (%) | (%) |
| 1 | None | 1.2 | 0 | 0 |
| 2 | Fe(acac)₃ | None | 0 | 0 |
| 3 | Fe(acac)₃ | 1.2 | 63 | 100 |
| 4 | Fe(acac)₃ | 1.8 | 64 | 100 |
| 5 | Fe(acac)₃ | 2.4 | 73 | 100 |
| 6 | FeCl₃ | 2.4 | 70 | 100 |
| 7 | Fe(dbm)₃ [symbol] | 2.4 | 7 | 43 |
| 8 | Fe(dpm)₃ [symbol] | 2.4 | 39 | 100 |
| 9 | Fe₂(SO₄)₃ | 2.4 | 0 | 0 |

[symbol]

dbm = [symbol] [symbol]
indicates data missing or illegible when filed

We turned to an examination of the scope of the reaction (Table 3). The arylative spirocyclization reaction is generally efficient with a range of commercially-sourced aryl Grignards. Only where chelating ortho-substituents are present (entry 13) was there a deleterious effect upon the yield (entry 13) In accord with 2a previous reports of the interaction of alkyl Grignard reagents with iron salts, the use of alkyl Grignard reagents in the spirocyclization reaction are not efficient, with only EtMgBr delivering products in detectable quantities (entry 14).

TABLE 3
Scope of Grignard component

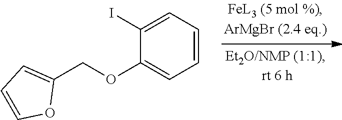 [SMILES: Ic1ccccc1OCc1ccco1]

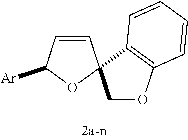

| Entry | Ar | Yield (%) | dr |
|---|---|---|---|
| 1 | 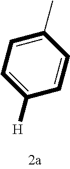 | Fe(acac) [symbol] : 73 FeCl [symbol] : 72 | 6:1 |
| 2 | 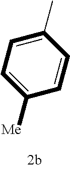 | Fe(acac) [symbol] :89 FeCl [symbol] :63 | 20:1 |
| 3 | 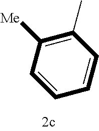 | Fe(acac) [symbol] : 76 FeCl [symbol] : 34 | trans-only |
| 4 | 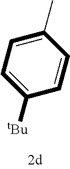 | Fe(acac) [symbol] : 39 FeCl [symbol] : 52 | 13:1 |
| 5 | 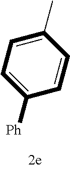 | Fe(acac) [symbol] : 96 FeCl [symbol] : 85 | 11:1 |
| 6 | 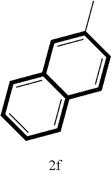 | Fe(acac) [symbol] : 91 FeCl [symbol] : 41 | trans-only |
| 7 | 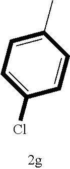 | Fe(acac) [symbol] : 49 FeCl [symbol] : 0 | |
| 8 | 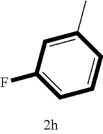 | Fe(acac) [symbol] : 88 FeCl [symbol] : 21 | 10:1 |
| 9 | 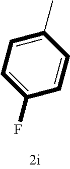 | Fe(acac) [symbol] : 75 FeCl [symbol] : 42 | trans-only |
| 10 | 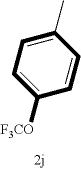 | Fe(acac) [symbol] : 39 FeCl [symbol] : 55 | 11:1 |
| 11 | 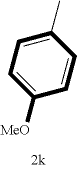 | Fe(acac) [symbol] : 74 FeCl [symbol] : 74 | 20:1 |
| 12 | 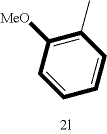 | Fe(acac) [symbol] : 0 FeCl [symbol] : 0 | — |
| 13 | 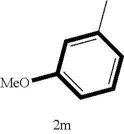 | Fe(acac) [symbol] : 88 FeCl [symbol] : 21 | 20:1 |
| 14 | 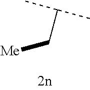 | Fe(acac) [symbol] : 36 FeCl [symbol] : 0 | |

[symbol]

indicates data missing or illegible when filed

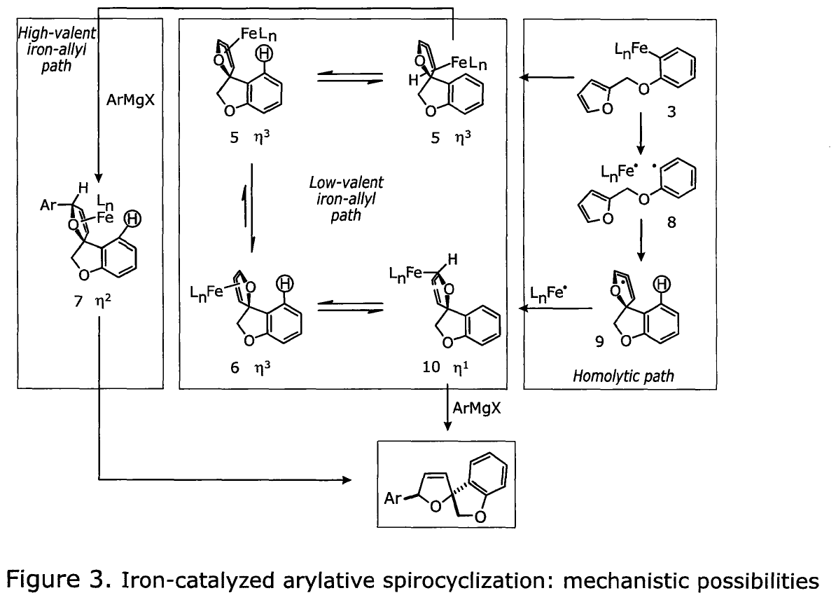

The mechanisms in play during iron-catalyzed cross-coupling reactions are complex and often not well-understood, and this arylative cyclization may proceeds by one of several possible pathways. Thus, a low-valent pathway may be in operation, proceeding via an intermediate σ-aryl iron intermediate³ 3 which cyclises to give an η³-allyl iron species 4 (FIG. 3) which will likely equilibrate to an η³-isomer 5. The latter may undergo isomerisation to avoid a repulsive interaction with an aromatic ring proton to give less-hindered isomer 6, which can be captured by Grignard to deliver the product after reductive elimination (and concomitant catalyst regeneration). Alternatively, if iron is high-valent the initially formed iron allyl may undergo anti-nucleophilic attack by Grignard to give η²-allyl iron complex 7, which will again deliver product, this time by decomplexation.

Finally, the reaction may involve radicals, if the initial formed σ-Fe—C bond undergoes homolysis to give aryl radical 8, which cyclises to give 9; radical recombination will give the same η¹-intermediate 10 as postulated for the low-valent path.

In summary, we have designed and implemented a novel iron-catalyzed cyclization-anion capture process which delivers spiroheterocycles in good yield. The mechanistic features of these reactions are a focus of our research at this time and these data will be disclosed elsewhere, in due course.

Various potential halogenated heterocycles and precursors thereof are shown in Scheme 1.

Scheme 2 shows the potential non-aromatic template rings and Scheme 3 shows various points of possible attachment.

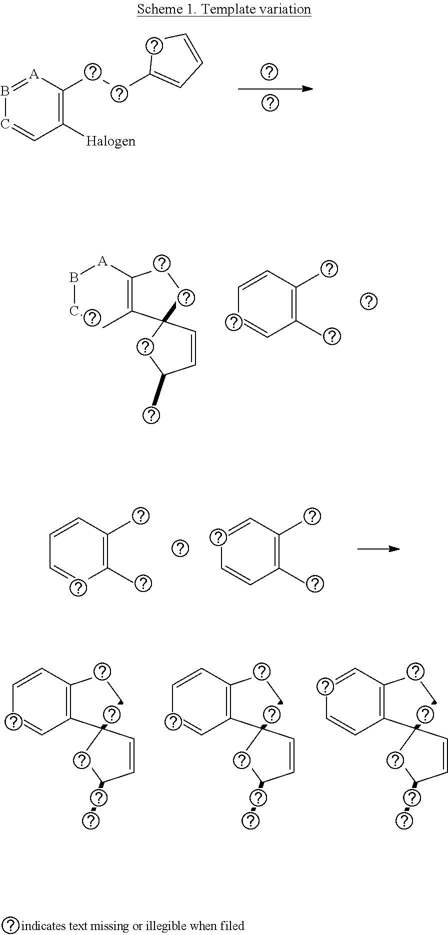

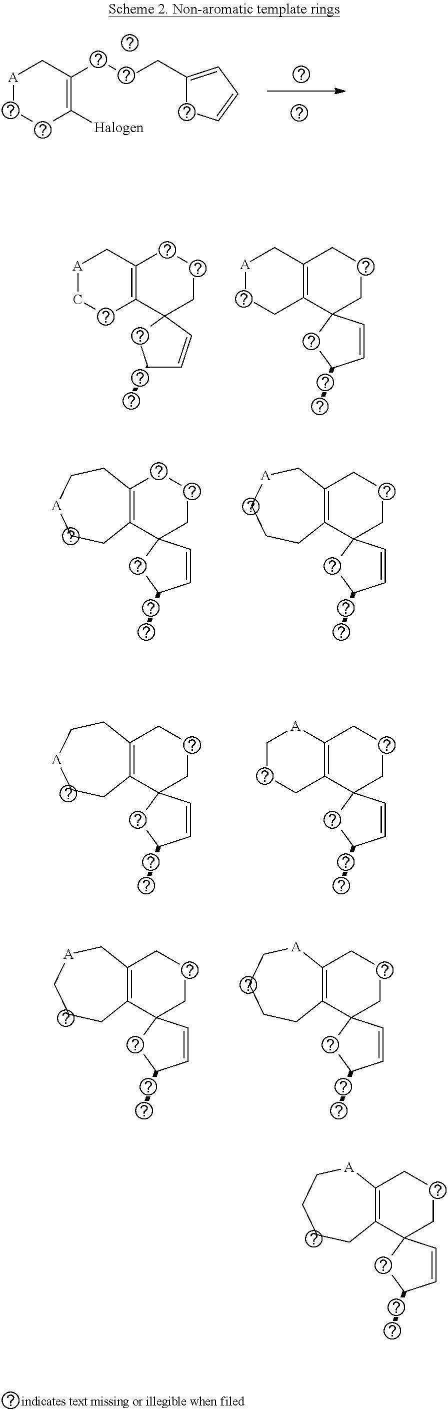

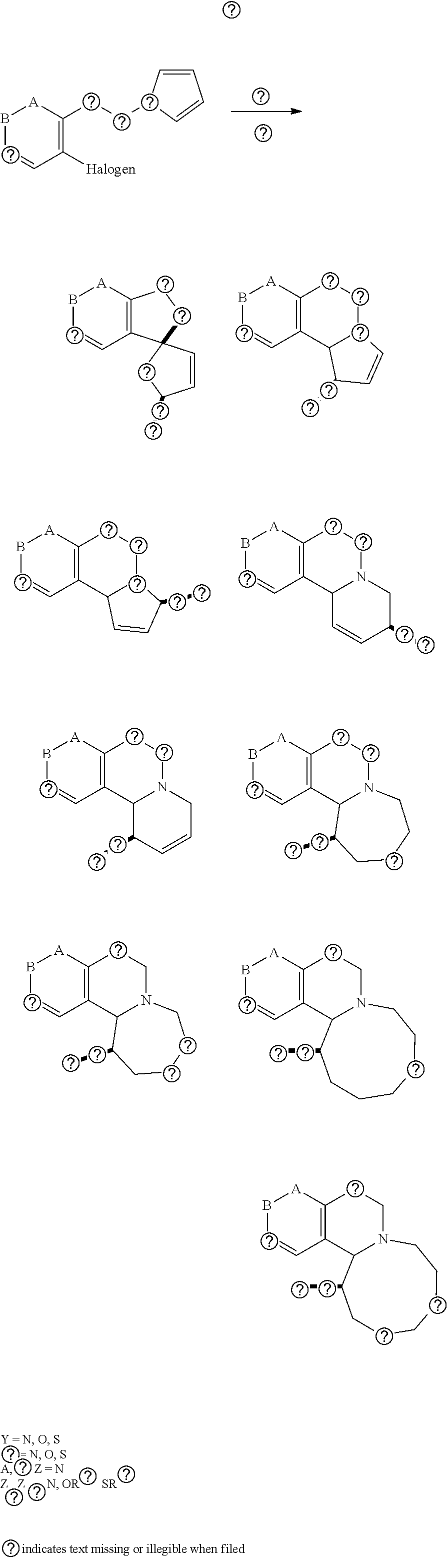

Scheme 4 below shows a synthetic route to spirocyclic ligands for asymmetric transformations.

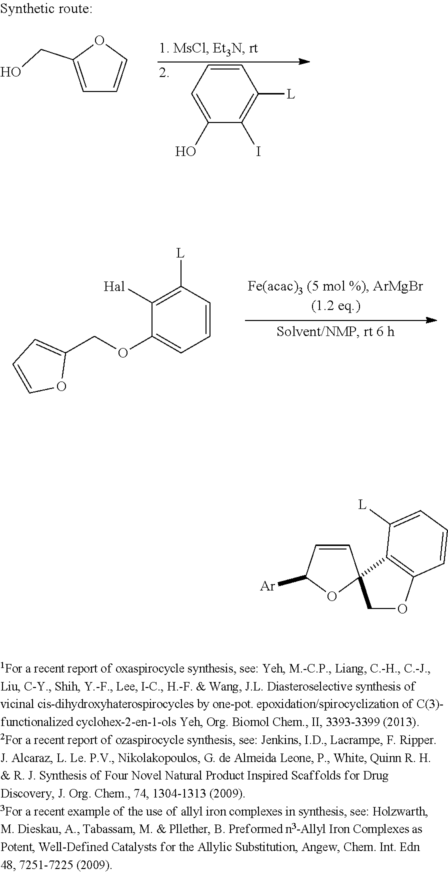

1. A process for the production of of spirocyclic compounds, said process including the steps of;
introducing or mixing at least one halogenated heterocyclic compound with iron or an iron containing complex; and
introducing at least one organometallic compound.

2. A process according to claim 1 wherein the iron containing complex includes iron (III).

3. A process according to claim 1 wherein the iron or iron containing complex is in a catalytic amount.

4. A process according to claim 3 wherein the process includes substantially 5 mol. % of iron (III) catalyst.

5. A process according to claim 4 wherein the process ues any one or any combination of Fe(acac)3, FeCl3, Fe(dbm)3 and/or the like.

6. A process according to claim 1 wherein the organometallic compound is an aryl and/or alkyl magnesium halide (Grignard reagent).

7. A process according to claim 6 wherein the organometallic compound is an organozinc reagent.

8. A process according to claim 1 wherein the halogenated heterocycle is derived from plants or plant related compounds.

9. Iron(III)-catalyzed arylative spirocyclization of at least one halogenated heterocyclic compound.

10. A spirocyclic ligand suitable for use in metal catalysed asymmetric transformations wherein said ligand has the general formula

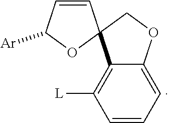

11. A ligand according to claim 10 wherein Ar is an aromatic group.

12. A ligand according to claim 10 wherein L is a Lewis base ligating group.

13. A metal catalysed asymmetric hydrogenation, hydroformylation, allylic substitution using ligands with the general formula [chemical structure]
